(19) United States
(12) Patent Application Publication
(10) Pub. No.: US 2022/0249512 A1
(43) Pub. Date: Aug. 11, 2022

(54) TOPICAL PHARMACEUTICAL COMPOSITIONS COMPRISING DILTIAZEM

(71) Applicant: S.L.A. Pharmca AG, Liestal (CH)

(72) Inventors: JUSTN SLAGEL, HERTFORDSHIRE (GB); EMIN A. CARAPETI, LONDON (GB)

(21) Appl. No.: 17/621,948

(22) PCT Filed: Jun. 27, 2019
(86) PCT No.: PCT/IB2019/000730

§ 371 Date: Dec. 22, 2021

(51) Int. Cl. A61K 31/554; A61K 9/00; A61K 9/06; A61K 45/06; A61K 31/4164; A61P 1/00

(52) U.S. Cl. CPC A61K 31/554; A61K 9/0031; A61P 1/00; A61K 45/06; A61K 31/4164; A61K 9/06

(57) ABSTRACT

The present invention provides a diltiazem preparation for treatment for anal fissures, hemorrhoids, or other benign anal disorders wherein the preparation is applied topically directly to the affected area and thereby reduces swelling, inflammation, pain and promotes healing.

### BACKGROUND OF THE INVENTION

### Technical Field

This invention relates to the use of a topical diltiazem composition for the treatment of anorectal disorders. The invention particularly relates to the treatment of anal fissures, anal cracks, anal fistulas, anal abscesses, anal pruritus and hemorrhoidal conditions.

### Related Art

Anal fissure is a split in the skin of the distal anal canal that is arbitrarily classified as acute or chronic according to presenting symptoms and age of the fissure. Chronicity is associated with a spasm of the internal anal sphincter muscle which increases the resting pressure in the anal canal which in turn reduces anodermal blood flow (Jones et al., 2002, Cross et al., 2008). Anatomical, angiographic, and blood flow studies have demonstrated that the blood supply of the anal epithelium is very poor in the posterior midline which is where the majority of anal fissures occur (Klosterhalfen et al., 1989; Lund et al., 1999). Sustained elevated resting anal pressure also causes a reduction in the number of transient relaxations of the internal anal sphincter (Farouk et al., 1994). Acute fissures are very common and most heal spontaneously, but a proportion progress to form a chronic linear ulcer in the anal canal and show great reluctance to heal without intervention.

Anal dilators have also been involved in treatment of anal fissures. Typically, a dilator of medium size was coated with anesthetic jelly and inserted into the anal canal before the passage of stool to prevent exacerbation of the symptoms during defecation. The procedure was inconvenient and success rate was low. The most common treatment, for chronic anal fissures is a lateral internal sphincterotomy, which involves surgery to the internal anal sphincter. This procedure, however, requires hospitalization and leads in a sizeable number of patients to impairment of continence.

Hemorrhoids are venous swellings of the tissues around the anus. Those above the dentate line (the point where the modified skin of the outer anal canal becomes gut epithelium), which usually protrude into the anal canal, are termed internal hemorrhoids, while those below this point are called external hemorrhoids. Due to internal pressure, internal hemorrhoids tend to congest, bleed and eventually prolapse; with external hemorrhoids painful thrombosis may develop.

Initial treatment of internal hemorrhoids may involve a high-fiber diet and avoidance of straining at stool, so bulk laxatives and fecal softeners may be indicated. Small bleeding hemorrhoids may be injected with a sclerosing agent such as oily phenol injection, or they may be ligated with rubber bands. More severe and prolonged prolapse generally requires surgery. Surgical excision to remove the clot is used for thrombosed external hemorrhoids.

A range of mainly topical drug treatments is available for symptomatic relief, but in many cases their value is a best unproven. Local anesthetics may be included to relieve pain, and corticosteroids may be used when infection is not present. Preparations containing either group of drugs are intended only for short-term use. Some preparations include heparinoids and other agents frequently included for their soothing properties include various bismuth salts, zinc oxide, hamamelis, resorcinol and peru balsam.

Currently, there are a number of topically applied formulations for the treatment of anorectal conditions, including ointments (creams, gels, jellies and pastes), foams, sprays and medicated pads. However, many of these have been relatively ineffective, while some, in particular, that become systemic are associated with unacceptable adverse effects. Thus, there is a particular need for a safe and effective topical treatment.

### SUMMARY OF THE INVENTION

In another aspect, the present invention provides a method of preventing or treating an anorectal disorder that includes topically applying, at least once a day to the mucosal surface of an anorectal region of a subject in need of such treatment, a therapeutically effective amount of a diltiazem containing topical composition of the present invention. The anorectal disorders treated with the compositions of the present invention include, but are not limited to, hemorrhoids, anal fissures, anal cracks, anal fistulas, anal abscesses, anal pruritus and other local anorectal lesions.

Without being bound by theory, it is believed that diltiazem is effective by lowering the anal resting pressure of the patient. This helps the fissures to heal. This reduction in anal pressure also allows better venous drainage which will allow the hemorroidal vascular cushions to heal. In the case of hemorrhoids, it is also thought that diltiazem will act to contract the longitudinal muscle of the anus, thereby pulling the hemorrhoidal cushions back into place.

The main object of the present invention is to provide a non-surgical treatment for anal fissures and/or hemorrhoids, or other benign anal disorders by providing a diltiazem preparation which reduces swelling, inflammation, and pain and promotes healing. The preparation is normally applied as an ointment directly to the affected area. The preparation helps to lubricate and add flexibility to the tissues, reduce swelling, inflammation, and pain and promote healing. Anal fissures are meant to include both acute and chronic fissures or ulcers. Any patient with persistent symptoms for more than two weeks is taken to have a chronic fissure in accordance with the invention. Hemorrhoids are meant to include both internal and external hemorrhoids and acute thrombosis of external hemorrhoid (TEM). Anorectal or perianal abscess (also known as anal/rectal abscess, perianal/perirectal abscess) is an abscess occurring adjacent to the anus, due to infection at one of the anal crypts of Morgagni. Anal abscess often leads to an anal fistula, which is the development of an infected channel within a gland between the anal canal and external skin near the anus or rectum. Anal pruritus is an irritation of the skin at the anus, associated with intensive urge to scratch the affected area.

Accordingly, in a preferred aspect of the invention there is provided the use of diltiazem or and pharmaceutically acceptable salts thereof in the preparation of a topical medicament for the treatment or prophylaxis of benign anal disorders, particularly in the treatment of anal fissures and hemorrhoids. The administration of diltiazem hydrochloride applied topically causes a reduction in the pain experienced by patients with chronic anal fissures. Clearly, the benefit of reducing pain causes an improved quality of life and potentially a shorter healing period.

Pharmaceutically acceptable salts of diltiazem include but is not limited to those formed with both organic and inorganic acids. Such acid addition salts will normally be pharmaceutically acceptable although salts of non-pharmaceutically acceptable salts may be of utility in the preparation and purification of the compound in question. Thus, preferred salts include those formed from hydrochloric, hydrobromic, sulphuric, citric, tartaric, phosphoric, lactic, pyruvic, acetic, succinic, oxalic, fumaric, maleic, oxaloacetic, methanesulphonic, ethanesulphonic, benzenesulphonic, and isethionic acids.

A suitable proportion of diltiazem in a topical or local composition for a beneficial effect is at least 0.5% w/w, such as 0.5% to 10% w/w, preferably 0.5% to 8% w/w, more preferably still 1% to 5% w/w, still more preferably 1% to 3%, and most preferably about 2% w/w. The diltiazem composition is suitably applied at least one time a day, 3 to 6 times, preferably 2 to 4 times daily. Dosage may include from about 2 mg/kg to about 25 mg/kg of diltiazem.

Pharmaceutical compositions adapted for topical administration in and/or around the anal canal may be formulated as ointments, creams, suspensions, emulsions lotions, solutions, pastes, gels, sprays, foam, oils, aerosols, suppositories or enemas and can be applied to the area by hand spreading the composition on the area, wipes comprising woven or non-woven fabric, cloth or tissue substrate and the wipe is impregnated and typically sealed into an enveloping sachet or pocket.

The topical compositions can comprise emulsifiers, preservatives, buffering agents and anti-oxidants. The compositions may also comprise steroids (e.g. present at 0.1 to 5% w/w) such as prednisolone, busenonide or hydrocortisone and locally acting anesthetics such as lignocaine. Typical components used in existing fissure or hemorrhoidal treatments which can also be used in topical compositions of the invention include, but is not limited to zinc oxide, benzyl benzoate, bismuth oxide, bismuth subgallate and Peru balsam.

The topical composition may further comprise skin penetrating agents, particularly the sulphoxides, such as dimethyl sulphoxide (DMSO). Amides, (DMA, DMF) pyrrolidones, organic solvents, laurocaprom (AZONE) and calcium thioglycollate are suitable alternative penetrants. The composition may also optionally contain a polyacrylic acid derivative, more particularly a carbomer. This would both act as a skin hydrating agent to aid penetration of the drug, but also an emulsifying agent. The carbomer will help emulsify the DMSO, thereby mitigating skin irritation and providing enhanced skin hydration. Propylene glycol may also be present in the composition to soften the skin, increase thermodynamic potential and aid skin penetration by the DMSO and thus the drug.

In a still further aspect, the present invention provides for a topical composition comprising an effective amount of a combination and/or admixture of diltiazem and metronidazole to reduce irritation and inflammation due to anal disorders. Preferably, the effective amount in the composition comprises a concentration of from about 10 to 25% w/w of metronidazole and 1% to 10% w/w diltiazem. A preferred composition further comprises propylene glycol and/or glycerol monostearate to enhance permeability.

In another aspect, the present invention provides a method for treating anus, rectum and perineal regions disorders comprising topically administering to a damaged area of a subject in need thereof, a therapeutically effective amount of a composition comprising from about 10% to 40% w/w of metronidazole and 1% to 10% w/w diltiazem or pharmaceutically acceptable salts thereof; and a pharmaceutically acceptable excipient or carrier.

In a still further aspect, the present invention relates to methods of controlling or alleviating pain by reducing the severity of inflammation and edema associated with damaged tissue in the anal and rectal area. wherein the method comprises: topically applying a therapeutically effective amount of a pharmaceutical composition comprising metronidazole and diltiazem and a pharmaceutically acceptable excipient. The topical composition is preferably in a form suitable for direct application to the damaged tissues. Suitable forms include ointment, lotion, gel, foam or cream. Notably, the combination metronidazole and diltiazem composition of the present invention relieves pain, reduces inflammation and edema and promotes wound healing.

Importantly, the topical compositions of the present invention can be applied to an animal following a surgical operation to the colon, rectum, anorectum or perianal region. The compositions may further comprise non-active agents comprising at least one component selected from the group consisting of emulsifiers, gelling agents, surfactants, preservatives, buffering agents, paraffin and solvents.

The dose of metronidazole for topical application is preferably between from about 7 mg/kg to about 125 mg/kg and more preferably between from about 2 mg/kg to about 25 mg/kg. The composition is usually applied between from 2 to 4 times daily and preferably 3 times daily.

In yet another aspect, the present invention relates to kits for the treatment of damaged tissue, wherein the kit includes packaging that contains a composition formulated for topical application and comprising at least an effective amount of metronidazole or salt thereof in a pharmaceutically acceptable carrier.

The composition of the present invention can be used topically by applying over an area to be treated. A typical method of use is to apply or rub the composition over the entire area, until the composition disappears. Several methods are available for the dispensing of the composition on the tissue damage including by physical means including applicator pads, swabs, or other devices intended to apply the composition in a thin film such as roller bottles, felt tip or sponge tip applicators.

Roll on bottles are especially advantageous. The roll on bottle greatly simplifies the dispensing of the composition on the tissue damage. No hand or finger rubbing is required. The movement of the roller ball on the surface massages the composition over the damaged tissue area. Further, the roller-ball provides a more precise control where the composition is to be applied, to avoid contact with hair around the anal area.

### DETAILED DESCRIPTION OF THE INVENTION

The present invention provides topical compositions comprising diltiazem and uses thereof for treating anorectal disorders, including hemorrhoids, anal fissures, anal cracks, anal fistulas, anal abscesses, anal pruritus and other local anorectal lesions.

Diltiazem is a benzothiazepine that acts as a calcium antagonist. Three main classes of calcium channel have been described and designated voltage-sensitive (cardiac and vascular smooth muscle), receptor-operated (cardiac and vascular smooth muscle) or stretch operated (some blood vessels). Voltage sensitive channels (voltage-gated calcium channels or VGCCs) are further sub-divided according to their activation and inactivation kinetics, their conductances, their ion specificities and their sensitivities to drugs and toxins. The family of L-type Ca²⁺ channels (L-channels) have high-affinity binding domains for diltiazem within their alpha₁,-subunits. Stereoselective, high-affinity binding of diltiazem induces blockade of channel-mediated inward Ca²⁺ currents causing muscle relaxation (Cross et al, 2008; Streissnig et al, 1998). The present invention shows that topically administration of diltiazem provides effective relaxation of anal sphincter tone.

As used herein, “hemorrhoids” mean swollen varicose veins in the mucous membrane inside or just outside the rectum. The composition and methods of the invention may be used to treat diseases of the anorectum which manifest one or more symptoms of itching, discomfort, pain and bleeding. Accordingly, references to use of the composition and/or methods of the invention for treating hemorrhoids is equally applicable to diseases of the anorectum manifesting one or more symptoms in common with hemorrhoids.

The term “pharmaceutical active”, “active pharmaceutical agent”, “active agent” and “drug” as used herein should be considered to have the same meaning.

The terms “effective amount” or “therapeutically effective amount” of an active agent as provided herein is defined as an amount of the agent at least sufficient to provide the desired therapeutic effect.

The term “w/w”, unless otherwise indicated, means weight of a given component or specified combination of components to total weight of the composition expressed as a percentage.

As used herein, the term “treat” or “treating” or “treatment” means to provide relief of one or more of the symptoms associated with anorectal disorders including but not limited to anal fissures, anal cracks, anal fistulas, anal abscesses, anal pruritus and hemorrhoidal conditions. The relief may be provided by ameliorating one or more symptoms, reducing the hemorrhoid, and/or healing affected tissues.

The term “petrolatum” refers to petroleum jelly, which is a mixture of the softer members of the paraffin or methane series of hydrocarbons, obtained from petroleum as an intermediate product in the distillation. Petrolatum is typically perceived as soothing when applied to the human skin.

Pharmaceutical Agents

The compositions of the present invention further comprise at least one additional pharmaceutically active agent in combination with the diltiazem, wherein the combination shows a synergistic effect such as an anesthetic agent, a vasoconstrictor, an antipruritic agent, an anti-inflammatory agent, a muscle relaxant, an astringent, a keratolytic agent, an antibiotic agent, an antiseptic agent, or a combination thereof. The compositions of the present invention may further comprise antioxidants. The compositions may further contain one or more protectant active ingredients, excipients and carriers. Pharmaceutically and dermatologically acceptable excipients and carriers as are known in the art may be included in the composition, in particular for maintaining the stability and sterility of the composition, and for promoting delivery, release and/or application of the active agent(s) to the body surface to which the composition is applied.

It is to be understood that the compositions may contain more than one active agent, and/or may be suitable for use in treating different anorectal or genital disorders. The pharmaceutically active agent and the dosage thereof is dependent upon the particular condition to be treated, the age of the subject and other factors evident to those skilled in the art. In an exemplified embodiment, the composition comprises an anesthetic agent and a vasoconstrictor. Anesthetic agents include, but are not limited to, pramoxine, procaine, lidocaine, tetracaine, dibucaine, prilocaine, phenacaine, benzyl alcohol, benzocaine, diperodon, dyclonine, dimethisoquin and combinations thereof. Exemplary anesthetic agent is pramoxine. Pharmaceutically acceptable salts of the aforementioned anesthetic agents may also be included in the composition of the invention. Suitable amounts of such anesthetic agents in the composition may be readily ascertained by one of ordinary skill in the art, and may range, for example, between 0.15% (w/w) and 25% (w/w).

Vasoconstrictors which are suitable for use in the invention include amphetamines, antihistamines, methylphenidate, mephedrone, oxymetazoline, phenylephrine, pseudoephedrine, psilocybin, phenylephrine hydrochloride, ephedrine sulphate, epinephrine, epinephrine hydrochloride, tetrahydrozoline hydrochloride, and combinations thereof. Suitable amounts of such vasoconstrictor agents in the composition may be readily ascertained by one of ordinary skill in the art, and may range, for example, between about 0.005% (w/w) and about 2% (w/w). Exemplary vasoconstrictor agent is phenylephrine HCl.

Antipruritic agents which are suitable for use in the invention include corticosteroids, camphor, juniper tar and menthol. The non-limiting examples of corticosteroids include hydrocortisone, fluocinolone, flurandrenolide, triamcinolone, fluticasone, and desonide. Antipruritic agents may further comprise corticosteroids such as tetrahydrocortisol, prednisone; prednisolone, fludrocortisone, 11-desoxycortisol, cortisone, corticosterone, paramethasone, betamethasone, dexamethasone, desoxycorticosterone acetate, desoxycorticosterone pivalate, fludrocortisone acetate, cortisol acetate, cortisol cypionate, cortisol sodium phosphate, cortisol sodium succinate, beclopmethasone dipropionate, betamethasone, betamethasone sodium phosphate and acetate, betamethasone dipropionate, betamethasone valerate, betamethasone benzoate, cortisone acetate, dexamethasone, dexamethasone sodium phosphate, dexamethasone acetate, fuprednisolone, meprednisone, methylprednisolone, methylprednisolone acetate, methylprednisolone sodium succinate, paramethasone acetate, prednisolone, prednisolone acetate, prednisolone sodium phosphate, prednisolone sodium succinate, prednisolone tebutate, prednisone, triamcinolone acetonide, triamcinolone diacetate, triamcinolone hexacotonide, desoximetasone, flumethasone pivalate, fluocinolone acetonide, fluocinonide, fluorometholone, halcinonide, and medrysone. Suitable amounts of antipruritic agents in the composition may be readily ascertained by one of ordinary skill in the art, and may range, for example, between about 0.1% (w/w) and about 5.0% (w/w).

Anti-inflammatory agents include salicylic acid, indomethacin, sodium indomethacin trihydrate, salicylamide, naproxen, colchicine, fenoprofen, sulindac, diflunisal, diclofenac, indoprofen and sodium salicylamide.

Muscle relaxants which are suitable for use in the invention include nitroglycerin, nifedipine, amlodopine, sildenafil, tizanidine, and baclofen, or salts thereof including, but not limited to, sildenafil citrate. Suitable amounts of such muscle relaxants in the composition may be readily ascertained by one of ordinary skill in the art, and may range, for example, between about 0.1% (w/w) and about 15% (w/w).

A topical composition of the present invention may further include an astringent. As used herein, an “astringent” refers to a substance that causes tissue (e.g., a hemorrhoidal) to contract and can optionally arrest secretion or control bleeding from tissue. Astringents which are suitable for use in the invention include, e.g., alum, tannic acid, calamine, witch hazel, zinc oxide, or a combination thereof. Suitable amounts of such astringents in the composition may be readily ascertained by one of ordinary skill in the art, and may range, for example, between about 2% (w/w) and about 50% (w/w).

A topical composition of the present invention my further include a surfactant. Non-limiting examples of possible non-ionic organic surfactants include polysorbates, such as polyoxyethylene sorbitan monolaurate (Tween 20), polyoxyethylene sorbitan monopalmitate (Tween 40), polyoxyethylene sorbitan monostearate (Tween 60) and polyoxyethylene sorbitan monooleate (Tween 80); glyceryl stearate; polyoxyethylene (POE) fatty acid esters, such as Myrj 45, Myrj 49, Myrj 52 and Myrj 59; poly(oxyethylene)alkylyl ethers, such as poly(oxyethylene) cetyl ether (Brij 52, Brij 56, Brij 58), poly(oxyethylene) palmityl ether, polyethylene oxide hexadecyl ether, polyethylene glycol cetyl ether, and the like; polyethoxylene castor oil derivatives, such as Cremophor EL, ELP and RH 40; PEG-6 octanoic/decanoic glycerides, such as Softigen 767 and the like; polyoxyethylene glycerol trioleate, such as but not limited to Tagat TO; decaglycerol mono/dioleate, such as Caprol PGE860 and the like; and a combination thereof.

Nonionic organic surfactants may further comprise sorbitan fatty acid esters, such as sorbitan monolaurate (Span 20), sorbitan monopalmitate (Span 40), sorbitan monooleate (Span 80), sorbitan monostearate (Span 60); mono/diglycerides of octanoic/dectanoic acids, such as but not limited to Imwitor-742, Imwitor-308, and a combination thereof.

Non-limiting examples of possible cationic surfactants include phosphatides, such as phosphatidyl choline and the like; quaternary ammonium cationic surfactants, such as hexadecyltrimethyl ammonium bromide and the like; pyrimidinium cationic surfactants, such as, but not limited to dodecyl pyridinium chloride; and a combination thereof.

Amphoteric surfactant may include lecithine, N-dodecyl alanine, cocamidopropyl amino betaine or a combination thereof. The type and the amount of surfactant may be determined by a person skilled in art so as to obtain the Hydrophile-Liphophile Balance (HLB) of the surfactant or the surfactant mixture suitable for the oil-in-water systems.

A topical composition of the present invention may further comprise a gelling agent to increases the aqueous phase viscosity when introduced in an aqueous phase. Without being bound to any mechanism of action, the topical composition in form of a gel comprises pharmaceutical agents primordially dissolved in the aqueous phase of the emulsion, finely dispersed in the continuous jelly phase and the silicone resin, primordially dissolved in the volatile solvent and finely dispersed in the aqueous phase of the emulsion, dispersed in the continuous jelly phase of the topical composition.

The gelling agent useful in a topical composition of the present invention may comprise hydroxypropyl cellulose, hydroxyethyl cellulose, hydroxypropyl methyl cellulose, methyl cellulose, ethyl cellulose, carboxymethyl cellulose, carbomer, carbomer copolymers, gelatin, aluminum monostearat, dextrin, sodium alginate, alginic acid, pectin, acacia, alginic acid, carrageenan, xanthan, tragacanth, magnesium aluminum silicate (Veegum®), bentonite, poloxamers (Pluronics®), polyvinyl alcohol, or mixtures thereof. Each possibility is a separate embodiment of the invention. In an embodiment, the gelling agents are cellulose derivatives. According to one embodiment, the gelling agent is hydropropyl methylcellulose. According to some embodiments, the gelling agent is not soluble is the volatile solvent and/or in the silicone oil phase of the emulsion. The amount of the gelling agent in the composition may be in a range from about 0.05% (w/w) to about 5.0% (w/w).

A topical composition of the present invention may further include a keratolytic agent. As used herein, a “keratolytic agent” refers to a substance that causes desquamation (loosening) and debridement or sloughing of the surface cells of the epidermis. Typically, the keratolytic agent used in the compositions of the present invention are pharmaceutically acceptable for topical use in humans. Suitable keratolytic agents include, but are not limited to, alcloxa, resorcinol, or a combination thereof. Suitable amounts of such keratolytic agents in the composition may be readily ascertained by one of ordinary skill in the art, and may range, for example, between about 0.1% (w/w) and about 5% (w/w).

Antibiotics for use in the invention are typically those suitable for topical application. The antibiotic(s) may be classified in one or more of the following groups: penicillins, cephalosporins, carbepenems, beta-lactam antibiotics, aminoglycosides, amphenicols, ansamycins, macrolides, lincosamides, glycopeptides, polypeptides, tetracylines, chloramphenicol, quinolones, fucidins, sulfonamides, sulfones, nitrofurans, diaminopyrimidines, trimethoprims, rifamycins, oxalines, streptogramins, lipopeptides, ketolides, polyenes, azoles, and echinocandins.

Specific examples of antibiotics used as an additional active agent having a synergistic effect with the diltiazem and which are suitable for use in the invention include: amikacin, aminosidine, paromomycin, chloramphenicol, ciprofloxacin, clindamycin, colistimethate-sodium, colistin, enfuvirtid, enoxacin, erythromycin, flucloxacillin, fosfomycin, fusafungin, gentamicin, levofloxacin, linezolid, mefloquin, metronidazol, mezlocillin, moxifloxacin, mupirocin, norfloxacin, ofloxacin, oxacillin, penicillin G, penicillin V, phenoxymethylpenicillin, phenoxymethylpenicillin-benzathin, pipemidinic acid, piperacillin, piperacillin+tazobactam, proguanil, propicillin, pyrimethamine, retapamulin, rifaximin, roxithromycin, sodium sulfacetamide, sulbactam, sulbactam+ampicillin, sulfadiazine, spiramycin, sultamicillin, tazobactam+piperacillin, teicoplanin, telithromycin, tigecyclin, vancomycin and combinations thereof.

Antiseptics which are suitable for use in the invention include, e.g., triclosan, phenoxy isopropanol, chlorhexidine gluconate, povidone iodine, and any combination thereof.

Antioxidative compounds may also be included in the composition, in particular, the antioxidative compounds collectively termed catechins. These include for example, epicatechin, epicatechin gallate, epigallocatechin gallate, and gallocatechin, as well as stereoisomers and enantiomers of these compounds and combinations thereof. Such compounds may be provided as synthetic compounds or in the forms of mixtures as components of plant extracts, in particular green tea extracts. Botanical products and extracts include those derived from peppermint, ginger horseradish, yarrow, chamomile, rosemary, capsicum, aloe vera, tea tree oil (melaleuca oil), among many others.

A topical composition of the present invention may further include protectant active ingredients. The protectant active ingredients can be selected from the group consisting of aluminum hydroxide gel, cocoa butter, aqueous solution of glycerin, hard fat, kaolin, lanolin, mineral oil, petrolatum, topical starch, white petrolatum, cod liver, shark liver oil, and a combination thereof. The protectant active ingredient and the dosage thereof is dependent upon the particular condition to be treated, the pharmaceutical active agents present in the composition and other factors evident to those skilled in the art.

A topical composition of the present invention may include one or more of the following additional ingredients: emulsifiers (e.g. anionic, cationic or nonionic), chelating agents, colorants, emollients, fragrances, surfactants, gelling agents, humectants, lubricants, moisturizers, preservatives, skin penetration enhancers, stabilizers, thickeners, and viscosity modifiers.

The compositions for use in the present invention may be stored in a container-applicator device for use in a single dose application or for use in repeated applications to the anus and rectum. Single dose applicators include those having breakable or removable seals that prevent moisture, including atmospheric moisture, from contacting the formulation.

A container-applicator may further comprise two parts: (1) a storage area or reservoir which holds the composition and protects it from air, water and contaminants; and (2) the applicator which generally comprises a specially shaped tip designed to aid in application of the composition to the anal and/or rectal mucosa.

Applicator tips can be of any of a number of shapes, sizes, and configurations. They may be fairly rigid and may be made out of any material which is compatible with the media formulation, such as plastic, excluding glass. The choice of a proper applicator tip for a given application will depend on factors such as the viscosity of the composition, the desired application rate of the composition, the nature of the anal disorder, and its severity.

Disorders of the anorectal region are commonly encountered among the general population, but are often inadequately unaddressed, since many patients delay or fail to seek medical attention due to embarrassment. Furthermore, many medications for such conditions fail to provide adequate relief and healing. In addition, many medications which are intended for treatment of conditions such as hemorrhoids and anal warts may be difficult to self-administer and are unsatisfactory due to their uncomfortable sensation after application.

The present invention provides compositions which are useful for effectively treating a variety of anorectal disorders including hemorrhoids, anal fissures, anal cracks, anal fistulas, anal abscesses, and anal pruritus, wherein the compositions provide enhanced therapeutic efficacy and are associated with improved patient compliance, as compared to prior art compositions.

The compositions of the present invention are applicable to both human patients and to non-human mammalian subjects such as in veterinary use, for example for treatment of canine, feline, equine, bovine, porcine and primate species.

The following examples illustrate certain embodiments of the invention but are not meant to limit the scope of the claims in any way. The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed.

Phase I Testing

Clinical Protocol to Assess the Pharmacokinetics of Topical Diltiazem in Patients With Anal Fissure

Pharmacokinetic data was generated in the patient population for diltiazem and its principle metabolites, N-monodesmethyldiltiazem, desacetyldiltiazem, desacetyl-N-monodesmethyldiltiazem, desacetyl-O-desmethyldiltiazem, and desacetyl-N, O-desmethyldiltiazem have been identified.

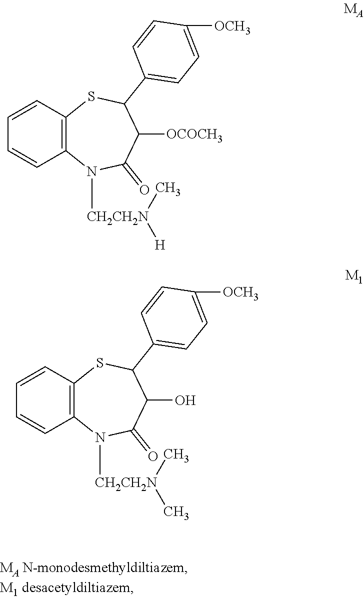

The finding of the above two structures were found following administration of diltiazem hydrochloride cream at strengths 2% w/w (n=4), 4% w/w (n=4) and 8% w/w (n=3). The formulation used had some minor differences to the amounts of propylene glycol and liquid paraffin when compared to the proposed commercial formulation. It also contained povidone. The formulation remains an oil/water emulsion with the drug substance in solution and these differences would not be expected to have an effect on the absorption profiles.

Patients with a confirmed diagnosis of an anal fissure, either acute or chronic, were selected as being representative of the adult population with anal fissure. The study was conducted in the European Union where the patient population was Caucasian (100%), with a mean age around 45 (range 26-57 years), divided 7:4 between male and female. As expected with topical application of drug product, rates of absorption varied considerably between individuals. After a single application (Day 1) the Cmax (mean±SD) were 1.88±0.80 ng/ ml, 2.68±2.03 ng/ml and 4.68±0.96 ng/ ml for the 2%, 4% and 8% preparations respectively. Maximum concentrations of the metabolites were generally less than 20% of those of the parent compound in molar terms.

Mean tmax for plasma diltiazem was similar for each of the three concentrations of diltiazem hydrochloride (5.50 h, 7.25 h, 6.67 h for 2%, 4% and 8% preparations respectively). Estimates of half-life (t½) are influenced by the continuing flux of diltiazem from the site of application and the effective (t½) is shown to increase with concentration (13.9 h, 16.34 h and 35.52h for 2%, 4% and 8% preparations respectively). On repeat dosing (three times daily) over a period of 4 days, the maximum concentrations of diltiazem and its metabolites increased up to 2-3 fold, relative to those observed for the single application for 2% and 4% preparations, whereas for 8% preparation, levels were 3.5-4.4 times higher.

Initial studies investigating the efficacy of diltiazem hydrochloride cream focused on fissure healing as the primary endpoint. Two important points have become apparent, firstly, pain is the principal presenting symptom and relief of pain is the most important goal of therapy from the patient's perspective and secondly, although complete healing is ultimately desired sometimes it is difficult to define. There is general acceptance that relief of pain is the more appropriate primary endpoint for clinical trials, with healing as a secondary endpoint.

With conservative therapy and given long enough, many fissures will heal with time. The proposed benefit of using diltiazem hydrochloride cream is to reduce the pain associated with anal fissure, allowing a greater degree of comfort during the healing period. This may also avoid the need for early surgery

Phase II Testing

Use of 0.2% Glyceryl Trinitrate and 2% Diltiazem Cream in the Treatment of Chronic Fissure in Ano: A Prospective, Multi-Centre, Double-Blind, Randomised Trial

Diltiazem hydrochloride 2% cream (n=31) twice daily for 6 weeks was shown to reduce overall anal fissure-related pain at a comparable rate to glyceryl trinitrate (GTN) 0.2% ointment (n=29). The diltiazem hydrochloride cream formulation used did not contain propylene glycol or glycerol monostearate and the absence of propylene glycol would be expected to affect the permeability of diltiazem and decrease the effectiveness of the product. As such, a preferred composition comprises propylene glycol and/or glycerol monostearate.

Patients with a confirmed diagnosis of chronic anal fissure defined as experiencing pain for more than 3 months or presence of a sentinel tag or visible muscle at the base of the fissure were selected as being representative of the majority of patients with chronic anal fissure. Assessment of benefit focussed on healing, but also included an assessment of pain. This was a randomised, double-blind, parallel group study, conducted in two centres within the European Union where the patient population had a mean age in the 40's (range 21-73 years), with similar distribution of males and females in the glyceryl trinitrate group and 20:11 males/females in the diltiazem group.

Considerable reductions in VAS scores for pain were obtained over the course of the study for both treatment groups (reduction in mean score from 62.8 mm to 26.7 mm for the diltiazem group compared with a reduction from 56.3 mm to 10.6 mm for the GTN group). There was no statistically significant difference between treatment groups in terms of VAS scores for pain (p≥0.24).

Phase III Testing

Randomised, Double-Blind, Placebo-Controlled Trial of the Safety and Efficacy of Diltiazem Hydrochloride Cream in Subjects with Anal Fissure

Diltiazem hydrochloride 2% cream (n=154) and diltiazem hydrochloride 4% cream (n=156) three times daily for 4 weeks have been shown to significantly reduce worst pain associated with or following defecation compared to placebo (n=155). Continued treatment with diltiazem hydrochloride cream (2% & 4%) through to 8 weeks also produced the key finding of an increase in the number of patients with healed anal fissures compared to placebo.

Patients were selected with a confirmed diagnosis of chronic anal fissure, defined as anal fissure-related pain associated with, or following, defecation experienced at least twice a week for the 4 weeks prior to screening, with an average pain score ≥3 on an 11-point numerical rating scale (NRS). During a one-week screening period, subjects recorded, on a daily basis, their scores for worst pain associated with, or following, defecation using the 11-point NRS. Only subjects having an average baseline score of ≥4 for the last three days on which defecation was recorded during screening were randomised. This was a randomised, double-blind, parallel group study, conducted in 27 centres in the European Union where the patient population was predominantly Caucasian (99.8%), with a mean age in the early 40′s (range 18-84) evenly distributed throughout the groups, with slightly more women than men (56%:44%). The primary endpoint was the change from baseline in average scores for worst pain associated with, or following, defecation at week 4. Secondary endpoints included the patient global impression of improvement (PGI-I), change from baseline in average scores for worst anal pain associated with or following defecation at each week, change from baseline in average scores for overall pain at each week, change from baseline in the SF-36 quality of life and proportion of subjects that had complete healing at week 8.

The mean baseline NRS scores (±SD) for worst pain associated with, or following, defecation were similar across the three treatment groups (4%: 6.40 (1.34); 2%: 6.21 (1.30); Placebo: 6.38 (1.33)). After 4 weeks treatment, there was a statistically significant reduction in pain score compared to placebo, with adjusted differences (97.5% CI; p-value) of −0.44 (<−0.06; p=0.0107) and −0.43 (<−0.06; p=0.0122) for the 4% and 2% treatment groups respectively.

The robustness of the response was confirmed in a number of sensitivity analyses. There was good correlation between all analyses, with consistent estimates of adjusted differences from placebo and statistical significance for both active treatment arms for all imputation methods tested. The reduction in NRS scores for worst pain associated with, or following, defecation for diltiazem hydrochloride cream treatment groups started to increase by week 2 compared to placebo and continued to increase throughout the 8-week treatment period. There were statistically significant reductions in pain score compared to placebo, for the 4% treatment group at weeks 4 to 8 (excluding week 6) and for the 2% treatment group at weeks 3 to 8. The magnitude of the adjusted difference from placebo in NRS score for worst pain associated with or following defecation at week 8 were −0.65 (p=0.0008) and −0.61 (p=0.0017) for the 4% and 2% treatment groups respectively.

In parallel with the results for the primary endpoint, NRS scores for overall pain decreased over the 8-week treatment period. There were statistically significant differences between the 4% treatment group and placebo at weeks 7 to 8 and for the 2% treatment group and placebo at weeks 3 to 8. Larger proportions of patients in the 4% and 2% treatment groups reported moderate or substantial improvements in the PGI-I compared to placebo. Pairwise comparisons versus placebo yielded p-values of 0.1317 and 0.0084 for the 4% and 2% treatment groups respectively.

There were also trends for improvements in the SF-36 quality of life instrument but with the exception of the physical component for the 2% treatment group these were not significant improvements. The proportion of subjects who had complete healing of the anal fissure by week 8 increased in the 4% w/w treatment group (32.7%) and 2% w/w treatment group (31.2%) compared to placebo (23.9%). The comparison for healing against placebo yielded p-values of 0.0184 and 0.0426 for the 4% w/w and 2% w/w treatment groups respectively.

### REFERENCES

The contents of the cited references are incorporated by reference herein for all purposes.

Cross K L R, Massey E J D, Fowler A L, Monson J R T. (2008) The management of anal fissure: ACPGBI Position Statement. Colorectal Dis; 10 (Suppl. 3): 1-7.

Farouk R, Duthie G S, MacGregor A B, Bartolo D C C. (1994) Sustained internal sphincter hypertonia in patients with chronic anal fissure. Dis Colon Rectum; 37(5): 424-429

Jones O M, Brading A F, Morrtensen N J McC. (2002) The physiology, pharmacology and therapeutic manipulation of the internal, sphincter. Can J Gastroenterol; 16:249-257.

Klosterhalfen B, Vogel P, Rixen H, Mittermayer C. (1989) Topography of the inferior rectal artery: a possible cause of chronic, primary anal fissure. Dis Colon Rectum; 32:43-52.

Lund J N, Binch C, McGrath J, Sparrow R A, Scholefield J H. (1999) Topographical distribution of blood supply to the anal canal. Br J Surg; 86:496-8.

Striessnig J, Grabner M, Mitterdorfer J, Hering S, Sinnegger M. J, Glossmann H. Structural basis of drug binding to L Ca²⁺ channels. Trends Pharmacol Sci. 1998;19:108-115.

That which is claimed is:

1. A method of preventing or treating an anorectal disorder; the method comprising topically applying a therapeutic amount in and around the anal region of a patient a topical composition, wherein the topical composition comprises:
diltiazem or a pharmaceutically acceptable salt of diltiazem; and
a pharmaceutically acceptable carrier.

2. The method of claim 1, wherein the topical composition is applied at least once a day.

3. The method of claim 1, wherein the anorectal disorder is selected from the group consisting of hemorrhoids, anal fissures, anal cracks, anal fistulas, anal abscesses, anal pruritus and other local anorectal lesions.

4. The method according to claim 1, wherein the topical composition further comprises at least one component selected from the group consisting of emulsifiers, preservatives, buffering agents, surfactants, gelling agents, paraffin and solvents.

5. The method of claim 1, wherein a therapeutic amount is a dosage from about 2 mg/kg to about 25 mg/kg.

6. The method of claim 1, wherein the topical composition formulated as ointments, creams, suspensions, emulsions lotions, solutions, pastes, gels, sprays, foam, oils, aerosols, suppositories or enemas.

7. The method of claim 1, wherein the composition is delivered in about a one (1) inch dosage of cream.

8. The method of claim 5, wherein the dosage is about 3 mg of diltiazem.

9. The method of claim 1, wherein the topical composition is applied three times daily.

10. The method of claim 9, wherein the applied three times daily was for a time period of eight weeks.

11. The method of claim 1, wherein the topical composition further comprises at least one additional pharmaceutically active agent selected from the group anesthetic agent, a vasoconstrictor, an antipruritic agent, an anti-inflammatory agent, a muscle relaxant, an astringent, a keratolytic agent, an antibiotic agent, an antiseptic agent, or a combination thereof.

12. The method of claim 1, wherein the topical composition further comprises propylene glycol and/or glycerol monostearate in an amount to increase permeability of diltiazem.

13. The method of claim 1, wherein the topical composition further comprises metronidazole.

14. The method of claim 13, wherein the topical composition comprises from about 10% to 40% w/w of metronidazole and 1% to 10% w/w diltiazem or pharmaceutically acceptable salts thereof.

15. A method of treating pain, the method comprising topically applying a therapeutic amount in and around the region of pain of a patient a topical composition, wherein the topical composition comprises:
diltiazem or a pharmaceutically acceptable salt of diltiazem; and
a pharmaceutically acceptable carrier.
